(19) United States
(12) Patent Application Publication
(10) Pub. No.: US 2025/0134499 A1
(43) Pub. Date: May 1, 2025

(54) BIOPSY NEEDLE ACCESSORY

(71) Applicant: The Johns Hopkins University, Baltimore, MD (US)

(72) Inventors: Robert Liddell, Lutherville, MD (US); Deborah I Weidman, Bedford, MA (US); Ashley Tsang, Hillsborough, CA (US); Gohta Aihara, Baltimore, MD (US); Tatiana Pereira, Baltimore, MD (US); Bibhav Poudel, Peoria, AZ (US); Jacob Maxwell Desman, Moorestown, NJ (US); Katherine Kovrizhkin, Baltimore, MD (US); Sean Darcy, Chula Vista, CA (US); Jinghua Zhang, Baltimore, MD (US); Shababa Matin, Baltimore, MD (US)

(21) Appl. No.: 18/693,749

(22) PCT Filed: Sep. 28, 2022
(86) PCT No.: PCT/US2022/077146

§ 371 Date: Mar. 20, 2024

Related U.S. Application Data
(60) Provisional application No. 63/249,680, filed on Sep. 29, 2021.

(51) Int. Cl. A61B 10/02

(52) U.S. Cl. CPC A61B 10/0233

(57) ABSTRACT

A biopsy needle accessory including a shaft with a first end, a second end, and a passageway extending between the first end and the second end. The shaft includes a circumferential wall, an aperture formed in the circumferential wall, and a ramp positioned within the passageway at the aperture. A biopsy needle is inserted through the passageway and extends through the aperture at an angle.

### CROSS-REFERENCE TO RELATED APPLICATIONS

This application claims priority to U.S. Provisional Patent Application No. 63/249,680, filed on Sep. 29, 2021, the entire contents of which are incorporated herein by reference.

### BACKGROUND

Conventional straight biopsy needles are utilized for conventional biopsies of tissue. For example, a lung biopsy is a medical procedure used to diagnose cancer and track a patient's treatment. Percutaneous lung biopsies are performed on over 150,000 patients in the U.S. yearly. This lung biopsy procedure includes inserting a straight biopsy needle directly through the skin, chest wall, and pleural space to sample nodules in the lung. Each insertion of a straight biopsy needle through the skin, chest wall, and pleural spaces creates a new hole, referred to herein as a puncture. Using conventional devices to reach a lung nodule, physicians typically require more than one puncture hole to navigate around obstacles (e.g., ribs, blood vessels, bronchi, etc.). In other words, it is difficult for a user to direct the conventional biopsy needle around structures to reach a desired target site and collect adequate tissue using only a single straight needle puncture. The additional puncture holes increase patient pain and the risk of patient complications (e.g., lung collapse, also referred to as pneumothorax) which may require further intervention (e.g., chest tube placement, overnight hospital stays), increased cost, or result in long term damage to the respiratory and cardiovascular system.

The difficulty in navigating conventional biopsy needles also results in unsuccessful biopsies that do not collect sufficient tissue to make a diagnosis, for example. In other words, because navigation of the biopsy needle is difficult, the user is unable to ensure an adequate sample is taken from the target tissue (i.e., the sample does not contain enough cells for analysis). This requires patients to repeat the biopsy procedure, which re-exposes patients to potential risks, delays in treatment, and cost.

Tissue biopsies are becoming increasingly utilized, especially for personalized medicine, for example, in which tissue samples are processed to analyze the genetic characteristic of a disease, thereby increasing the need for improved biopsy needle navigation.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

One aspect of the present disclosure provides a biopsy needle accessory including a shaft with a first end, a second end, and a passageway extending between the first end and the second end. The shaft includes a circumferential wall, an aperture formed in the circumferential wall, and a ramp positioned within the passageway at the aperture.

In some embodiments, the passageway is configured to receive a biopsy needle such that the biopsy needle extends through the aperture.

In some embodiments, the aperture is formed in the circumferential wall at the second end.

In some embodiments, the shaft defines a longitudinal axis extending between a first end and a second end.

In some embodiments, the ramp is oriented at an angle with respect to the longitudinal axis.

In some embodiments, the angle is within a range of 0 degrees and 30 degrees. In some embodiments, the angle is within a range of 5 degrees and 30 degrees.

In some embodiments, the angle is 10 degrees.

In some embodiments, the shaft is configured to be inserted into a guide needle.

In some embodiments, the shaft includes an axial end surface at the second end.

In some embodiments, the biopsy needle accessory further includes a handle coupled to the first end of the shaft, wherein the handle includes a handle passageway coupled to the passageway of the shaft.

In some embodiments, the handle passageway includes a funnel portion.

In some embodiments, the handle includes a boss coupled to the first end of the shaft.

In some embodiments, the handle includes an indicia aligned with the ramp.

In some embodiments, the biopsy needle accessory further includes a seal positioned within the handle.

In some embodiments, the handle includes a first portion and a second portion, and the seal is positioned between the first portion and the second portion.

In some embodiments, the biopsy needle accessory further includes a first cap having a first cap proximal end and a first cap distal end, wherein the first cap proximal end is coupled to the handle.

In some embodiments, the first cap includes a first seal positioned within a bore.

In some embodiments, the biopsy needle accessory further includes a second cap having a second cap proximal end and a second cap distal end, wherein the second cap distal end is coupled to the handle, and wherein the second cap includes a second seal.

In some embodiments, the second cap includes a stem at least partially received within the handle passageway.

In some embodiments, the biopsy needle accessory further includes a first air-tight cap coupled to a first handle end. In some embodiments, the biopsy needle accessory further includes a second air-tight cap coupled to a second handle end.

Another aspect of the present disclosure provides a kit including a biopsy needle and an accessory including a handle and a shaft. The shaft includes an aperture formed in a circumferential wall and a ramp positioned at the aperture. The biopsy needle is receivable within the shaft and configured to extend through the aperture.

In some embodiments, the kit further includes a guide needle with a guide needle handle, and the shaft is receivable within the guide needle.

In some embodiments, the kit further includes a first cap with a first seal and a second cap with a second seal. The first cap is positioned between the guide needle handle and the accessory handle. The accessory handle is positioned between the first cap and the second cap.

In some embodiments, the kit further includes a guide needle cap and a seal positioned in the guide needle cap.

In some embodiments, the kit further includes a seal positioned in the handle.

In some embodiments, the kit further includes a stylet, and the stylet is receivable within the shaft.

Other aspects of the disclosure will become apparent by consideration of the detailed description and accompanying drawings.

### BRIEF DESCRIPTION OF THE DRAWINGS

The accompanying figures and examples are provided by way of illustration and not by way of limitation. The foregoing aspects and other features of the disclosure are explained in the following description, taken in connection with the accompanying example figures (also “FIG.”) relating to one or more embodiments.

FIG. 1 is a perspective view of a guide needle and a biopsy needle accessory.

FIG. 2 is an exploded view of the guide needle and the biopsy needle accessory of FIG. 1.

FIG. 3 is another perspective of the exploded view of FIG. 2.

FIG. 4A is a cross-sectional view of the guide needle and the biopsy needle accessory of FIG. 1

FIG. 4B is an enlarged partial view of the cross-sectional view of FIG. 4A.

FIG. 4C is an enlarged partial view of the cross-sectional view of FIG. 4A.

FIG. 5 is a partial perspective cross-sectional view of the guide needle and biopsy needle accessory of FIG. 1.

FIG. 6 is a side view of a biopsy needle extending from a guide needle.

FIG. 7A is a side view of a biopsy needle extending from a biopsy needle accessory, where the biopsy needle accessory is extending from a guide needle.

FIG. 7B is a cross-sectional view of FIG. 7A.

FIG. 8A is a cross-sectional view of a biopsy needle partially extending through a biopsy needle accessory.

FIG. 8B is a cross-sectional view of a biopsy needle partially extending through a biopsy needle accessory.

FIG. 9 is a cross-sectional view of a shaft portion of a biopsy needle accessory.

FIG. 10 is a cross-sectional view of a shaft portion of a biopsy needle accessory.

FIG. 11 is a perspective view of a guide needle, a biopsy needle accessory, and a stylet.

FIG. 12 is an exploded view of the guide needle, biopsy needle accessory, and stylet of FIG. 11.

FIG. 13 is another exploded view of the guide needle, biopsy needle accessory, and stylet of FIG. 11.

FIG. 14A is a cross-sectional view of the guide needle, biopsy needle accessory, and stylet of FIG. 11.

FIG. 14B is an enlarged partial view of the cross-sectional view of FIG. 14A.

FIG. 15 is a partial perspective cross-sectional view of the guide needle, biopsy needle accessory, and stylet of FIG. 11.

FIG. 16A is a cross-sectional view of a straight ramp.

FIG. 16B is a cross-sectional view of a curved ramp.

FIG. 16C is a cross-sectional view of a ramp with a curved portion leading into a straight slope portion.

FIG. 16D is a cross-sectional view of a ramp with two curved portions and a straight slope portion positioned between the two curved portions.

FIG. 17 is a side view of an accessory shaft illustrating an aperture.

FIG. 18 is a top view of the accessory shaft and aperture of FIG. 17.

Before any embodiments are explained in detail, it is to be understood that the invention is not limited in its application to the details of construction and the arrangement of components set forth in the following description or illustrated in the following drawings. The invention is capable of other embodiments and of being practiced or of being carried out in various ways.

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “an element” means at least one element and can include more than one element.

“About” and “approximately” are used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that an apparatus comprises components A, B, and C, it is specifically intended that any of A, B or C, or a combination thereof, can be omitted and disclaimed singularly or in any combination.

Recitation of ranges of values herein is merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

According to one aspect of the present disclosure, a biopsy needle accessory that integrates with a conventional workflow and biopsy system, such that a biopsy needle can extend in a straight path or at an angled path.

As described herein, the accessory increases the safety and procedural efficacy of percutaneous lung procedures. Although the accessory is described herein as being utilized with a lung biopsy, the accessory is configured for utilization with a standard biopsy system and procedures used across a variety of organ systems. For example, the standard biopsy system that this accessory supports is utilized for a variety of biopsies, including, but not limited to, kidney, prostate, liver, lymph nodes, spleen, pancreas, and thyroid biopsy procedures. Similar to a lung biopsy, biopsy needle users in general face similar navigation and tissue efficacy challenges. Therefore, the described accessory can also be utilized for all these biopsy procedures. In some embodiments, the accessory detailed herein is used with other types of needle devices for other procedures including, but not limited to, drug delivery and seed placement. In some embodiments, the accessory is used with thin needle brachytherapy, for example.

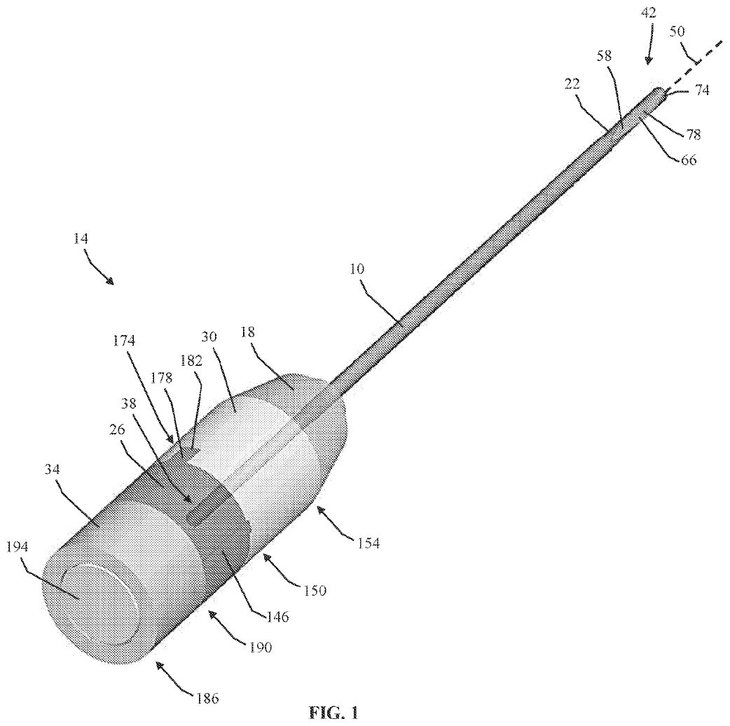

With reference to FIG. 1, a guide needle 10 and a biopsy needle accessory 14 (i.e., a biopsy needle attachment) are illustrated. In the illustrated embodiment, a guide needle handle 18 is coupled to the guide needle 10. In some embodiments, the guide needle 10 and the guide needle handle 18 are a trocar. In some embodiments, the trocar is a standard-of-care 17 G trocar. In other embodiments, the guide needle is any suitable size.

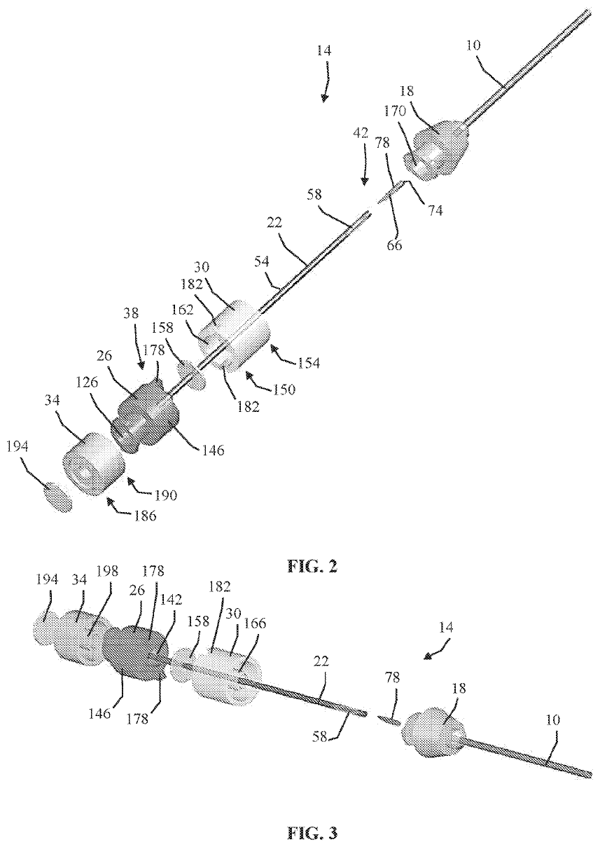

With reference to FIGS. 2 and 3, the biopsy needle accessory 14 includes a shaft 22, a handle 26, a first cap 30, and a second cap 34. The shaft 22 includes a first end 38 (i.e., a proximal end), a second end 42 (i.e., a distal end), and a passageway 46 extending between the first end 38 and the second end 42. In the illustrated embodiment, the shaft 22 is a hollow shaft. The shaft 22 defines a longitudinal axis 50 that extends between the first end 38 and the second end 42. In some embodiments, the shaft 22 is made of stainless steel. In some embodiments, the shaft 22 is made of stainless steel 304, stainless steel 316, titanium, cobalt chrome, or carbon-fiber-reinforced polyetheretherketone. In some embodiments, the shaft 22 is made of any suitable medical grade metal or still material. As discussed in further detail herein, the shaft 22 is configured to be inserted into the guide needle 10. In other words, the shaft 22 is at least partially received within the guide needle 10. In some embodiments, the shaft 22 is used alone without the guide needle 10.

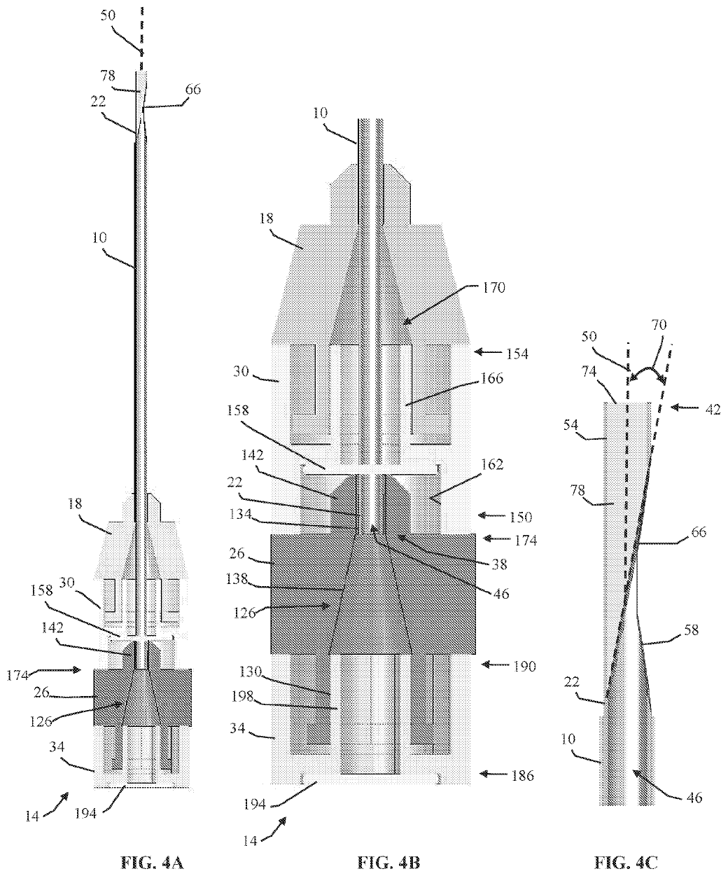

With reference to FIGS. 1, 4A and 4C, the shaft 22 includes a circumferential wall 54 and an aperture 58 formed in the circumferential wall 54. In the illustrated embodiment, the aperture 58 is formed in the circumferential wall 54 at the second end 42 of the shaft 22. In other words, the aperture 58 is closer to the second end 42 than the first end 38. As discussed in further detail herein, the passageway 46 is configured to receive a biopsy needle 62 such that the biopsy needle 62 extends through the aperture 58 at an angle (e.g., FIGS. 7A and 8B). In some embodiments, the biopsy needle 62 is a 20 G biopsy needle. In other embodiments, the biopsy needle is any suitable size.

With continued reference to FIG. 4C, the shaft 22 includes a ramp 66 positioned within the passageway 46 at the aperture 58 (i.e., near or adjacent to the aperture 58). The ramp 66 is oriented at an angle 70 with respect to the longitudinal axis 50. In other words, the ramp 66 intersects the longitudinal axis 50 at the angle 70. In some embodiments, the angle 70 is within a range of approximately 0 degrees (e.g., no ramp) and approximately 30 degrees. In some embodiments, the angle 70 is within a range of approximately 5 degrees and approximately 30 degrees. In the illustrated embodiment, the angle 70 is approximately 10 degrees. In other embodiments, the angle 70 is approximately 5 degrees. In other embodiments, the angle 70 is approximately 15 degrees. In other embodiments, the angle 70 is approximately 0 degrees. The angle 70 is representative of the angle at which the biopsy needle 62 will extend from the aperture 58 of the shaft 22.

With continued reference to FIG. 4C, the shaft 22 includes an axial end surface 74 at the second end 42 that closes the second end 42 of the shaft 22. In other words, the distal end surface 74 of the shaft 22 is closed. In the illustrated embodiment, the axial end surface 74 is formed as an insert 78 that is received within the second end 42 of the shaft 22. In some embodiments, the insert 78 is secured within the second end 42 the hollow shaft 22 by an interference fit. In the illustrated embodiment, the ramp 66 is formed on the insert 78. The separately formed insert 78 advantageously improves the manufacturability of the shaft 22 and the accessory 14 overall. In some embodiments, the size of the shaft 22 is 18 G, or any other suitable size.

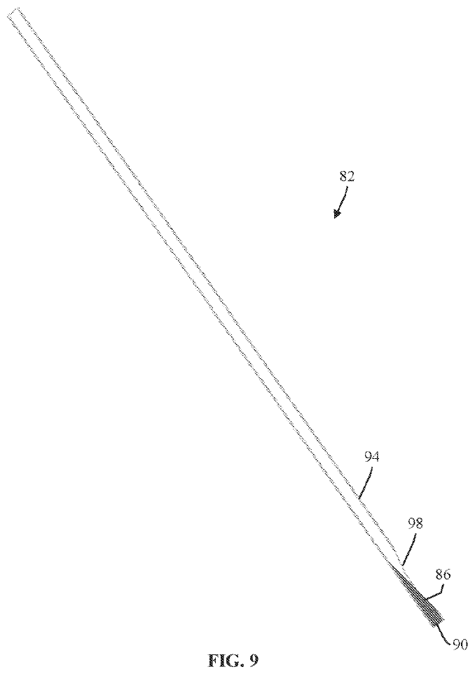

With reference to FIG. 9, a shaft 82 for the accessory 14 according to another embodiment is illustrated. The shaft 82 is a single manufactured piece (i.e., a unitary piece). In the illustrated embodiment, a ramp 86 and an axial end surface 90 are contiguously formed with a circumferential wall 94 including an aperture 98. In other words, no insert (similar to the insert 78) is needed for the shaft 82.

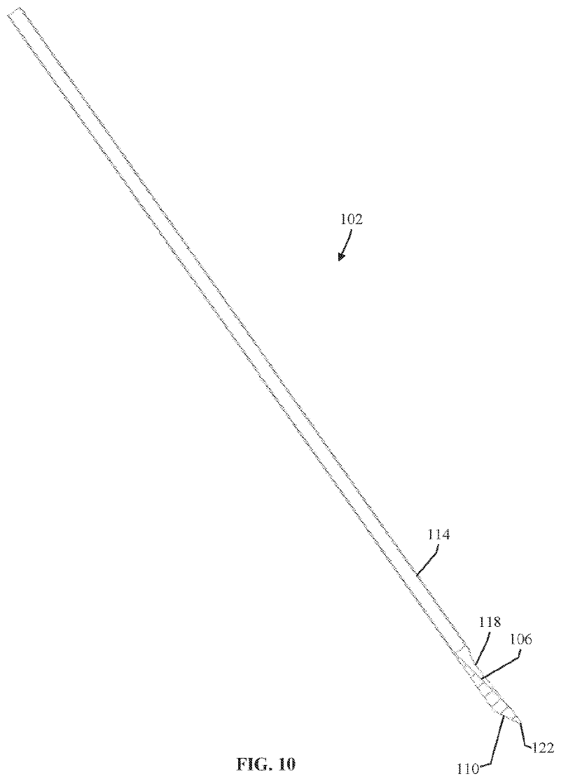

With reference to FIG. 10, a shaft 102 for the accessory 14 according to another embodiment is illustrated. The shaft 102 is a single manufactured piece (i.e., a unitary piece). In the illustrated embodiment, a ramp 106 and an axial end surface 110 are contiguously formed with a circumferential wall 114 including an aperture 118. In the illustrated embodiment, the axial end surface 110 is angled to form a tip 122 (e.g., a sharp point). In other words, the tip 122 of the shaft 102 is sharp and capable of piercing through skin, organs, tissue, etc. In other embodiments, the axial end surface 110 is flat. In some embodiments, the shaft of the accessory 14 is interchangeable with any one of the shafts (e.g., shaft 22, 82, 102) described herein.

With reference to FIG. 4B, the handle 26 of the accessory 14 is coupled to the first end 38 of the shaft 22 and the handle 26 includes a handle passageway 126 coupled to the passageway 46 of the shaft 22. In other words, the handle passageway 126 is joined with and in fluid communication with the passageway 46 of the shaft 22. In the illustrated embodiment, the handle passageway 126 includes a first cylindrical portion 130, a second cylindrical portion 134, and a funnel portion 138 positioned between the first cylindrical portion 130 and the second cylindrical portion 134. In the illustrated embodiment, the handle 26 includes a boss 142 coupled to and surrounding the first end 38 of the shaft 22. In other words, the boss 142 is a mounting boss that receives the shaft 22. In some embodiments, the handles are pre-attached via a manufacturing process to the guide needles and/or accessory.

With reference to FIG. 1, the handle 26 in the illustrated embodiment includes an indicia 146 that is aligned with the ramp 106 to indicate to a user which direction the aperture 58 is pointing. In the illustrated embodiment, the indicia 146 is a straight line that indicates where the aperture 58 and the ramp 66 are positioned relative to the circumferential wall 54. In some embodiments, the indicia 146 visually indicates the opening direction of the aperture 58. In some embodiments, the indica 146 is a mark or arrow. In some embodiments, the indicia 146 tactilely indicates the opening direction of the aperture 58. In some embodiments, the indica 146 is a raised ridge and/or a lowered ridge.

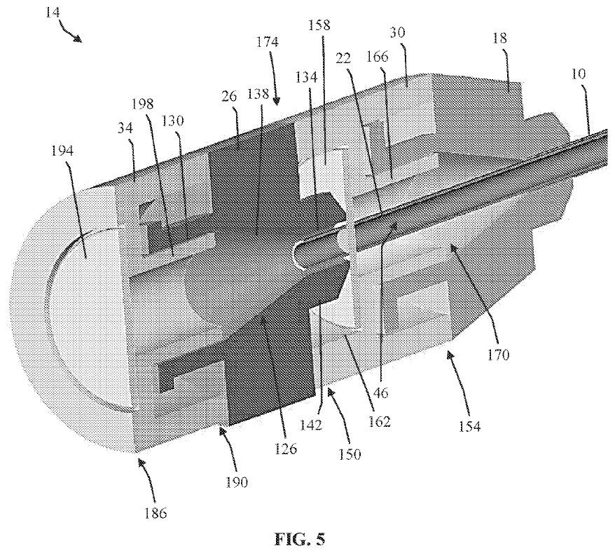

With reference to FIG. 4B and 5, the first cap 30 includes a first cap proximal end 150 and a first cap distal end 154. In the illustrated embodiment, the first cap proximal end 150 is coupled to the handle 26 and the first cap distal end 154 is coupled to the guide needle handle 18. The first cap 30 includes a first seal 158 positioned within a bore 162. The first cap distal end 154 includes a stem 166 configured to be at least partially received within a passageway 170 of the guide needle handle 18. The first cap proximal end 150 is coupled to the handle 26 with a rotationally locking interface 174. In the illustrated embodiment, the interface 174 includes a key portion 178 formed on the handle 26 configured to be received within a corresponding slot 182 formed on the first cap 30. When assembled, the interface 174 prevents relative rotational movement between the first cap 30 and the handle 26 (and correspondingly the shaft 22 also). In other embodiments, the interface 174 is any suitable mechanical interface that prevents relative rotation between components.

With continued reference to FIGS. 4B and 5, the second cap 34 includes a second cap proximal end 186 and a second cap distal end 190. The second cap distal end 190 is coupled to the handle 26. The second cap 34 includes a second seal 194 positioned at the second cap proximal end 186. With reference to FIG. 4B, the second cap 34 includes a stem 198 (similar to the stem 166) at least partially received within the handle passageway 126. In the illustrated embodiment, the stem 198 is removably coupled to the first cylindrical portion 130 of the handle passageway 126.

As explained in further detail herein, the first cap 30 and the second cap 34 are air-tight caps. In other words, the seals 158, 194 in the first cap 30 and the second cap 34 prevent air from passing through the guide needle 10 or the shaft passageway 46 when there is no biopsy needle or stylet inside the passageway 46. The air-tight caps 30, 34 advantageously prevent air from entering the pleural space, for example, which can lead to a lung collapse (pneumothorax). The air-tight caps 30, 34 can also prevent the leakage of blood and other body fluids in procedures such as in the liver or kidney. In some embodiments, the seals 158, 194 are made of a rubber. In some embodiments, the seals 158, 194 are made of synthetic elastomer, such as silicone. In some embodiments, the seals 158, 194 are made of any suitable elastomer material. In some embodiments, the seals include an aperture, a thinner layer of material, and/or a valve.

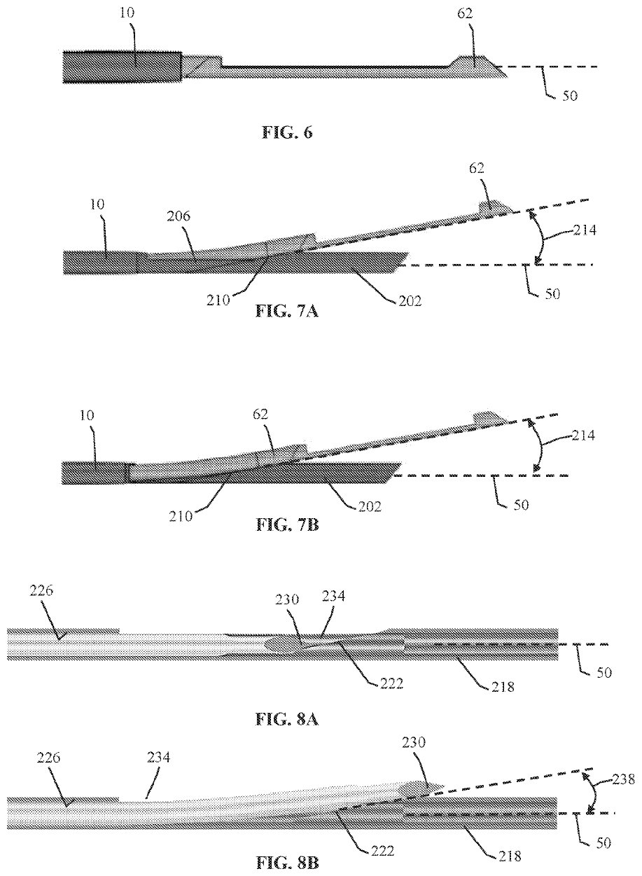

With reference to FIG. 6, the biopsy needle 62 is shown extending from the guide needle 10 in a straight line, along the axis 50. With reference to FIG. 7A, a shaft 202 according to one embodiment is illustrated extending from the guide needle 10. The biopsy needle 62 extends from an aperture 206 of the shaft 22. In particular, the shaft 202 includes a ramp 210 (FIG. 7B) which causes the biopsy needle 62 to deflect and extend at an angle 214 with respect to the guide needle 10 and the axis 50. In other words, the biopsy needle 62 contacts the ramp 210 and extends through the aperture 206 at the angle 214.

With reference to FIGS. 8A-8B, the progression of the biopsy needle 62 moving through a shaft 218 according to one embodiment. The biopsy needle 62 deflects upon contact with a ramp 222 formed in the shaft 218. Specifically, FIG. 8A illustrates the biopsy needle 62 extending through a passageway 226 of the shaft 218 before a tip 230 of the biopsy needle 62 contacts the ramp 222. In other words, before the tip 230 contacts the ramp 222, the biopsy needle 62 is oriented in a straight line (i.e., along the center longitudinal axis 50). FIG. 8B illustrates the biopsy needle 62 extending from an aperture 234 in the shaft 218 and deflected by the ramp 222 at an angle 238 with respect to the longitudinal axis 50. In other words, when the tip 230 has passed over the ramp 222, the biopsy needle 62 extends from the aperture 234 at the angle 238.

In operation, the guide needle 10 is inserted into a patient or subject and the first cap 30 is attached to the guide needle handle 18 to create an air-tight seal through the guide needle 10. Then, the shaft 22 is inserted into the guide needle 10, passing through the seal 158 of the first cap 30. The accessory 14 is rotated about the insertion axis so the aperture 98 is oriented towards the target site and the accessory 14 is moved forward and backwards along an insertion axis to reach a target site. The second cap 34 is then attached to the handle 26 to establish an air-tight seal through the passageway 46. Then, the biopsy needle 62 is inserted through the shaft 22 and deflects at the ramp 66 to extend at the angle 70 with respect to the guide needle 10. The ramp 70 is oriented by a user to deflect the biopsy needle 62 to a target location.

During a procedure, the user may remove the shaft 22 with a first ramp 66 (e.g., at 10 degrees) and replace it with a second shaft with a second ramp (e.g., at 5 degrees) to quickly accommodate changing navigation requirements. In this case, when the shafts are replaced, no additional puncture is created because the guide needle remains in place. In other words, the shafts 22, 102, 202, and 218 are interchangeable with the guide needle 10 to offer seamless transition to different biopsy needle navigation. Once the tissue sample has been taken by the biopsy needle, the biopsy needle 62, the shaft 22 and the guide needle 10 are removed from the patient or subject, leaving only a single incision in the patient or subject (through which the guide needle punctured).

During some procedures, the user can rotate the accessory 14 about the longitudinal axis 50 to take multiple samples. Specifically, the user takes a first sample with a biopsy needle while the accessory shaft 22 is in a first orientation, takes out the biopsy needle and retrieves the sample. Then, the user can rotate the accessory shaft 22 about the longitudinal axis 50 and takes a second sample with a biopsy needle. The biopsy needle itself can move forward or backwards in the accessory 14 to reach even more samples of the tissue. Each time the biopsy needle is removed and/or each time the accessory shaft is replaced, the airtight seals disclosed herein prevent airflow and a possible pneumothorax. In some embodiment, the seals are integrated within handles. All of these additional samples are collected with the accessory 14, which only creates a single puncture.

As such, the biopsy needle accessory 14 described herein increases the safety and procedural efficacy of biopsy procedures. The accessory 14 enables a user (e.g., an interventional radiologist) to insert a biopsy needle (e.g., biopsy needle 62) at a variety of angles to more easily reach a target biopsy site with only a single puncture. By facilitating access to various target sites, the accessory 14 simultaneously increases the amount of tissue obtained in a single needle pass and decreases risks (e.g., pneumothorax, bleeding, etc.) and other procedural complications.

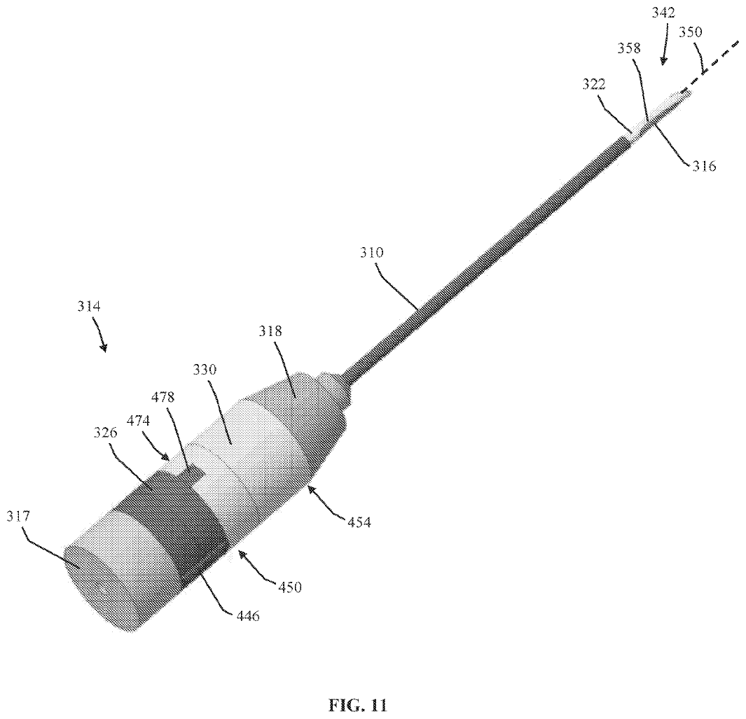

With reference to FIGS. 11-15, a guide needle 310, a biopsy needle accessory 314 and a stylet 316 are illustrated. With reference to FIG. 11, the stylet 316, the accessory 314, and the guide needle 310 are shown assembled together. In the illustrated embodiment, a guide needle handle 318 is coupled to the guide needle 310. In the illustrated embodiment, a stylet handle 317 is coupled to the stylet 316.

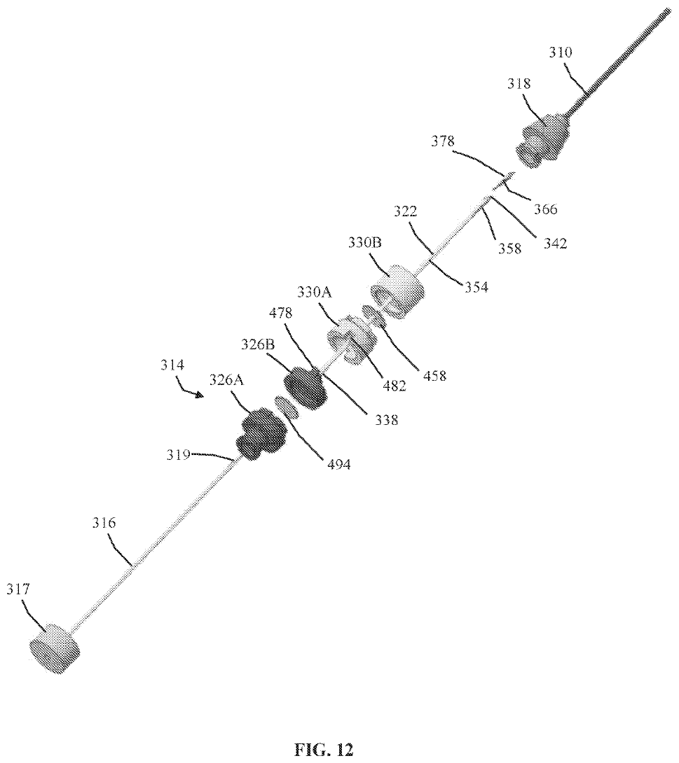

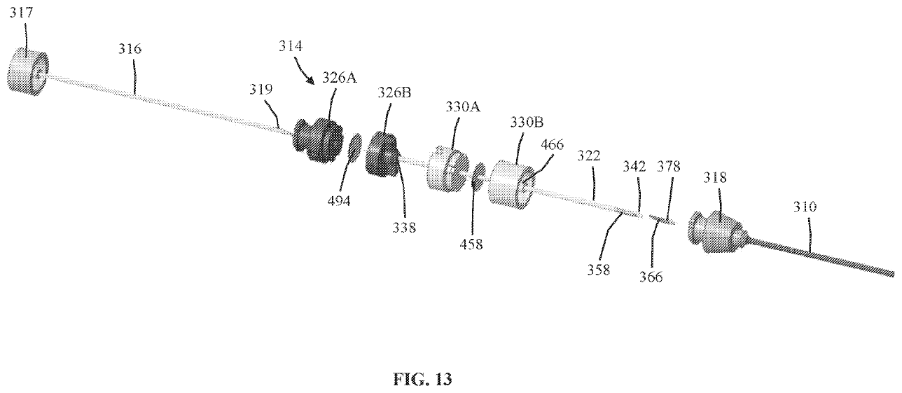

With reference to FIGS. 12 and 13, the biopsy needle accessory 314 includes a shaft 322, a handle 326, a cap 330 (e.g., a guide needle cap). In some embodiments, the cap 330 is considered part of the biopsy needle accessory 314. In other embodiments, the cap 330 is considered part of the guide needle 310. The shaft 322 includes a first end 338 (i.e., a proximal end), a second end 342 (i.e., a distal end), and a passageway 346 extending between the first end 338 and the second end 342. In the illustrated embodiment, the shaft 322 is a hollow shaft. The shaft 322 defines a longitudinal axis 350 that extends between the first end 338 and the second end 342. In some embodiments, the shaft 322 is configured to be inserted into the guide needle 310. In other words, the shaft 322 is at least partially received within the guide needle 310. In other embodiments, the shaft 322 is used without the guide needle 310 and/or can replace the guide needle 310

With reference to FIGS. 11, 17, and 18, the shaft 322 includes a circumferential wall 354 and an aperture 358 formed in the circumferential wall 354. In the illustrated embodiment, the aperture 358 is formed in the circumferential wall 354 at the second end 342 of the shaft 322. In other words, the aperture 358 is closer to the second end 342 than the first end 338. In the illustrated embodiment, the aperture 358 includes a wide end 359A and a tapered end 359B positioned opposite the wide end 359A. In some embodiments, the aperture 358 is a custom teardrop shape. The aperture 358 formed in the circumferential wall 354 creates a cutout 355 with an arcuate profile 356 (e.g., side profile shown in FIG. 17). As discussed herein, the passageway 346 is configured to receive a biopsy needle such that the biopsy needle extends through the aperture 358 at an angle. In the illustrated embodiment, the aperture 358 shape minimizes the opening from which the biopsy needle comes out to provide maximum support of the biopsy needle upon exit.

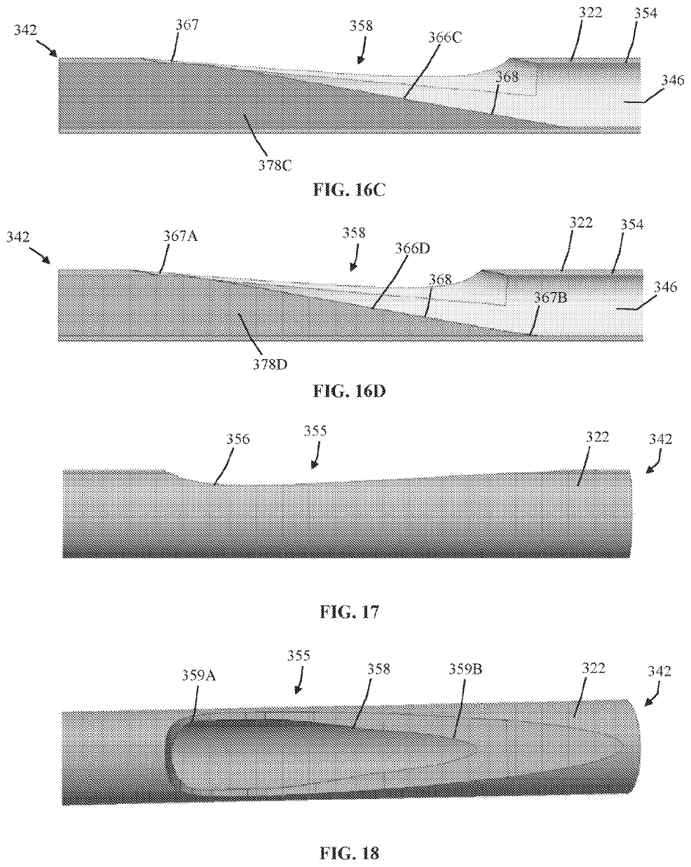

With reference to FIGS. 12 and 13, an insert 378 with a ramp 366 is positioned within the shaft 322 at the second end 342. With reference to FIGS. 16A-16D, inserts 378A-D having ramps 366A-D of various geometries are illustrated and received within the second end 342 of the shaft 322. FIG. 16A illustrates a straight ramp 366A (e.g., a linear ramp). FIG. 16B illustrates an arcuate ramp 366B (e.g., a curved ramp). FIG. 16C illustrates a ramp 366C with a curved portion 367 leading into a straight slope portion 368. FIG. 16D illustrates a ramp 366D with a first curved portion 367A, a second curved portion 367B and a straight slope portion 368 positioned between the two curved portions 367A, 367B. In some embodiments, the ramp includes rounded corners or end points. In some embodiments, the ramp is concave. In other embodiments, the ramp is convex. In some embodiments, the ramp is planar or flat. In some embodiments, the ramp is concave with a defined radius (of any suitable radius). In some embodiments, the ramp is curved at the endpoints with a defined radius. In some embodiments, both endpoints are curved. In some embodiments only one endpoint is curved. Curvature in the ramp advantageously reduces friction of the biopsy needle inserted through the accessory. In other words, an arcuate ramp allows the biopsy needle to come out through the aperture casily and smoothly. In some embodiments, the ramp has a biocompatible coating (also to reduce friction of the biopsy needle inserted through the accessory). With reference to FIG. 11, the handle 326 in the illustrated embodiment includes an indicia 446 that is aligned with the ramp 366 to indicate to a user which direction the aperture 358 is pointing.

The stylet 316 is configured to be received within the shaft 322. In some embodiments, the stylet 316 is a solid rod (stainless steel, or any medical grade steel, for example) that fits inside the accessory 314 and has a ramp 319 that complements the ramp 366 of the accessory 314. In other words, a distal end of the stylet 316 complements the shape of the ramp 366 and fits into the aperture 358. In some embodiments, the stylet 316 is inserted into the accessory 314 when the accessory 314 is being inserted into the body to minimize tissue damage due to the aperture 358. In some embodiment, the stylet handle 317 is attached to the top of the stylet shaft during manufacturing, and as a system can screw onto the accessory handle 326 by the user upon accessory insertion into the body. The stylet 316 can then later be detached (e.g., screwed off) to allow passage of a biopsy needle through the accessory 314.

With reference to FIGS. 12, 13, 14A, 14B, the accessory 314 includes a seal 494 positioned within the handle 326. In the illustrated embodiment, the handle 326 includes a first portion 326A and a second portion 326B, and the seal 494 is positioned between the first portion 326A and the second portion 326B. In other words, the seal 494 is integrated into the handle 326. In some embodiments, the seal 494 is integrated with a prolonged press-fit. In some embodiments, the seal 494 is integrated with the handle 326 and a biopsy needle is used with just the accessory 314 (e.g., no guide needle).

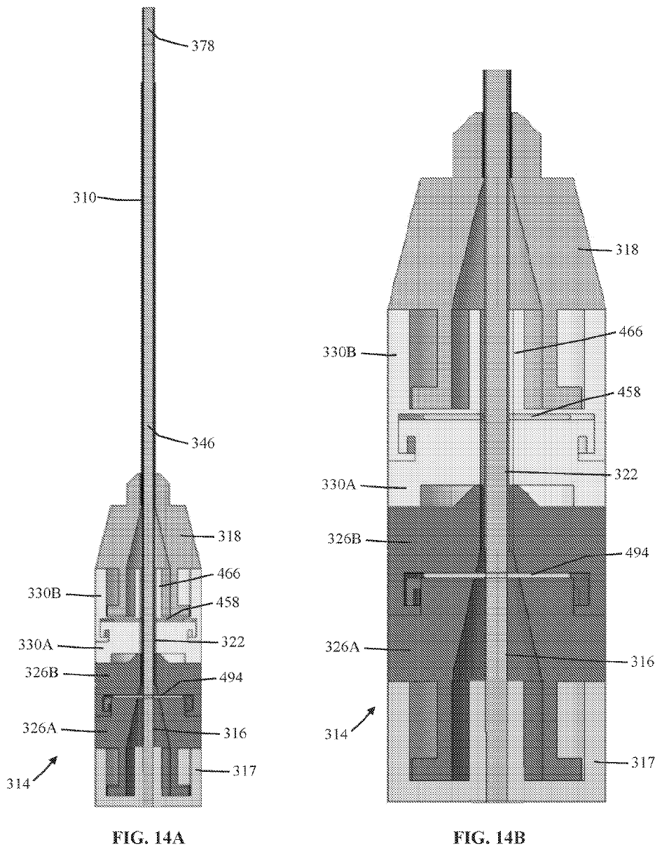

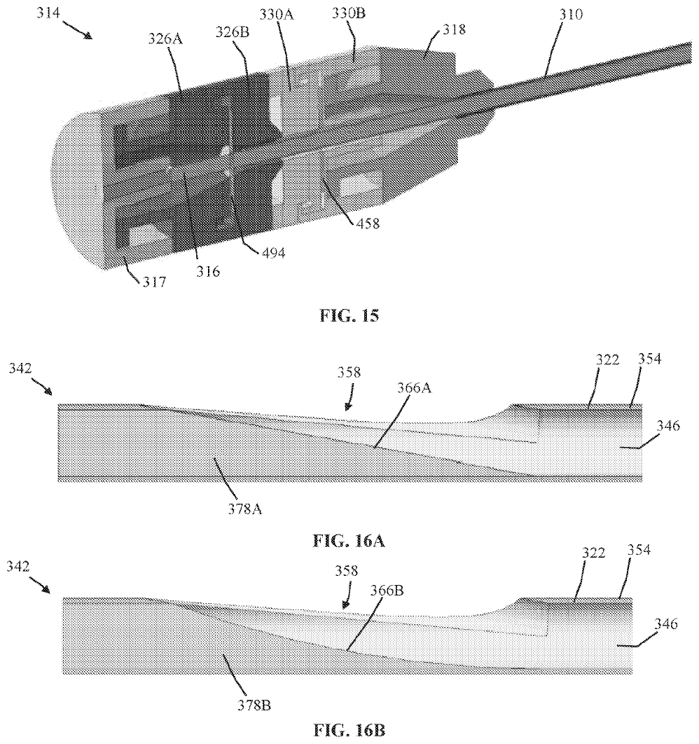

With reference to FIGS. 14A, 14B and 15, the cap 330 includes a cap proximal end 450 and a cap distal end 454. In the illustrated embodiment, the cap proximal end 450 is coupled to the handle 326 and the cap distal end 454 is coupled to the guide needle handle 318. The cap 330 includes a seal 458 positioned within the cap 330. In the illustrated embodiment, the cap 330 includes a first cap portion 330A and a second cap portion 330B, and the seal 458 is positioned between the first portion 330A and the second portion 330B. In other words, the scal 458 is integrated into the cap 330.

With continued reference to FIGS. 14A, 14B, and 15 the cap distal end 454 includes a stem 466 configured to be at least partially received within the guide needle handle 318. The cap proximal end 450 is coupled to the handle 326 with a rotationally locking interface 474. In the illustrated embodiment, the interface 474 includes a key portion 478 formed on the handle 326 configured to be received within a corresponding slot 482 formed on the cap 330. When assembled, the interface 474 prevents relative rotational movement between the cap 330 and the handle 326 (and correspondingly the shaft 322 also). In other embodiments, the interface 474 is any suitable mechanical interface that prevents relative rotation between components.

The cap 330 and the accessory handle 326 provide air-tight seals. In other words, the seals 458, 494 in the cap 30 and the handle 326 prevent air from passing through the guide needle or the shaft passageway when there is no biopsy needle or stylet inside the passageway. In some embodiments, the seals are disc-shaped. In some embodiments, the seal includes a valve (e.g., a leaf valve with leaflets, a one-way check valve, or other suitable valve) that puckers and allows the needle to pass through and closes back to retain a flat surface upon needle exit to prevent air from leaking from the body, through the needle, and to the atmosphere. In some embodiments, the seal includes a very small aperture in the center of it to allow for case of puncture with minimal air escape. The integrated air-tight seals advantageously prevent air from entering the plural space of the lung, for example, which can cause lung collapse (pneumothorax). They can also prevent the leakage of blood and other body fluids in procedures such as in the liver or kidney. In some embodiments, the seals 458, 494 are made of a rubber or a synthetic elastomer, such as silicone. In some embodiments, the seals are integrated directly into handles and no separate caps are included. For example, in some embodiments, a kit includes a biopsy needle, a guide needle with guide needle handle with integrated seal, and shaft with accessory handle with integrated seal.

In some embodiments, a kit includes a biopsy needle (e.g., biopsy needle 62) and an accessory (e.g., accessory 14, accessory 314, etc.) with an accessory handle and a shaft. The shaft includes an aperture (e.g., aperture 58, aperture 358) formed in the circumferential wall 54 and the ramp (e.g., ramp 66, ramp 366A, ramp 366B, etc.) is positioned at or near the aperture. The biopsy needle is receivable within the shaft and is configured to extend through the aperture 58. In some embodiments, the kit further includes a guide needle (e.g., guide needle 10, guide needle 310, etc.) and the shaft is receivable within the guide needle. In some embodiments, the kit further includes a stylet (e.g., style 316), wherein the stylet is receivable within the shaft.

In some embodiments, the kit further includes the first cap 30 with the first seal 158 and the second cap 34 with the second seal 194. The first cap 30 is positioned between the guide needle handle 18 and the accessory handle 26, and the accessory handle 26 is positioned between the first cap 30 and the second cap 34.

In some embodiments, the kit further includes a guide needle cap (e.g., cap 330) and an integrated seal (e.g., seal 458) positioned within the guide needle cap. In some embodiments, an integrated seal (e.g., seal 494) is positioned within the accessory handle.

One skilled in the art will readily appreciate that the present disclosure is well adapted to carry out the objects and obtain the ends and advantages mentioned, as well as those inherent herein. The present disclosure described herein are exemplary embodiments and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

Various features and advantages are set forth in the following claims.

What is claimed is:

1. A biopsy needle accessory comprising:
a shaft with a first end, a second end, and a passageway extending between the first end and the second end;
wherein the shaft includes a circumferential wall, an aperture formed in the circumferential wall, and a ramp positioned within the passageway at the aperture.

2. The accessory of claim 1, wherein the passageway is configured to receive a biopsy needle such that the biopsy needle extends through the aperture.

3. The accessory of claim 1, wherein the aperture is formed in the circumferential wall at the second end.

4. The accessory of claim 1, wherein the shaft defines a longitudinal axis extending between a first end and a second end.

5. The accessory of claim 4, wherein the ramp is oriented at an angle with respect to the longitudinal axis.

6. The accessory of claim 5, wherein the angle is within a range of 5 degrees and 30 degrees.

7. The accessory of claim 6, wherein the angle is 10 degrees.

8. The accessory of claim 1, wherein the shaft is configured to be inserted into a guide needle.

9. The accessory of claim 1, wherein the shaft includes an axial end surface at the second end.

10. The accessory of claim 1, further including a handle coupled to the first end of the shaft, wherein the handle includes a handle passageway coupled to the passageway of the shaft.

11. The accessory of claim 10, wherein the handle passageway includes a funnel portion.

12. The accessory of claim 11, wherein the handle includes a boss coupled to the first end of the shaft.

13. The accessory of claim 10, wherein the handle includes an indicia aligned with the ramp.

14. The accessory of claim 10, further includes a seal positioned within the handle.

15. The accessory of claim 14, wherein the handle includes a first portion and a second portion, and wherein the seal is positioned between the first portion and the second portion.

16. The accessory of claim 1, further including a first cap having a first cap proximal end and a first cap distal end, wherein the first cap proximal end is coupled to the handle.

17. The accessory of claim 16, wherein the first cap includes a first seal positioned within a bore.

18. The accessory of claim 17, further including a second cap having a second cap proximal end and a second cap distal end, wherein the second cap distal end is coupled to the handle, and wherein the second cap includes a second seal.

19. The accessory of claim 18, wherein the second cap includes a stem at least partially received within the handle passageway.

20. The accessory of claim 1, further including a first air-tight cap coupled to a first handle end.

21. A kit comprising:
a biopsy needle; and
an accessory including a handle and a shaft, the shaft includes an aperture formed in a circumferential wall and a ramp positioned at the aperture; and
wherein the biopsy needle is receivable within the shaft and configured to extend through the aperture.

22. The kit of claim 21, further including a guide needle with a guide needle handle; wherein the shaft is receivable within the guide needle.

23. The kit of claim 22, further including a first cap with a first seal and a second cap with a second seal; wherein the first cap is positioned between the guide needle handle and the handle; and wherein the handle is positioned between the first cap and the second cap.

24. The kit of claim 22, further including a guide needle cap and a seal positioned in the guide needle cap.

25. The kit of claim 21. further including a seal positioned in the handle.

26. The kit of claim 21. further including a stylet, wherein the stylet is receivable within the shaft.
